(19) United States
(12) Patent Application Publication
(10) Pub. No.: US 2011/0052646 A1
(43) Pub. Date: Mar. 3, 2011

(54) POLYMER ADHESIVE FILM FOR DIRECTED CELLULAR GROWTH

(76) Inventor: Darnell Kaigler, SR., Detroit, MI (US)

(21) Appl. No.: 12/871,377

(22) Filed: Aug. 30, 2010

Related U.S. Application Data
(60) Provisional application No. 61/238,019, filed on Aug. 28, 2009.

(51) Int. Cl. A61K 9/00; B05D 5/00; B05D 3/10; A61P 17/02

(52) U.S. Cl. 424/400; 427/2.31; 216/37

(57) ABSTRACT

The described embodiments relate to a polymer adhesive film having a micro-pattern arranged on a first surface of the polymer adhesive film for application to wounded tissue to promote directional cell growth. The micro-pattern is sized to allow cells of the wounded tissue to grow directionally within the micro-pattern to promote rapid and complete healing.

### FIELD OF THE INVENTION

Embodiments described herein relate generally to a polymer adhesive film for use in closing wounds, and more particularly, to a polymer adhesive film including micro-patterns to direct cellular growth to facilitate rapid wound healing.

### BACKGROUND OF THE INVENTION

To prevent infection and promote healing, it is a common practice to close a wound with sutures and protect the surrounding damaged tissues with a dressing or other covering. For example, healing of oral tissue after oral surgery (i.e., surgical tooth extraction) may be hindered by normal masticatory action, tongue movements during speech, and salivary fluid flow. Additionally, debris from food deposits can delay the clotting cascade or disrupt an established clot, and thus, interfere with and delay healing. Therefore, after oral surgery, the surgical incision is typically sutured to attain primary closure of the wound in order to promote healing. However, suturing techniques can be cumbersome, are time consuming, and require a high degree of skill to perform correctly. Furthermore, sutures may not have the necessary strength to hold a wound closed, particularly in the mouth where the wound may be disturbed by the normal functional processes described above. An additional drawback to the use of sutures is that the patient often needs to have them removed at a later date.

In addition to sutures, a dressing, such as gauze or a periodontal pack is commonly placed on the surgical site. The dressing may be applied to direct pressure to the wound in order to help stop bleeding, protect against contaminants, and act as a temporary physical barrier to the oral environment. However, a dressing made of an absorbent material, such as cotton, has a limited ability to prevent moisture and saliva from reaching the surgical site in that it may become saturated. Such a dressing is usually only effective for a few hours after surgery. Dressings used on wounds inside and outside of the oral environment suffer from additional drawbacks, such as: need for frequent removal and changing; difficult to attain adhesion of the dressing to the wound; inadequate mechanical properties; and difficult application.

It may also be desirable to apply a therapeutic formulation at the wound or surgical site to promote healing. However, topical formulations applied directly or integrated with commonly used dressings are quickly lost due to moisture and mechanical action, and additionally, these formulations are not capable of penetrating skin or mucous membranes. If used in combination with a dressing, therapeutic formulations have several other drawbacks including lack of biodegradability, damage or irritation to the skin during removal of the dressing, covalent bonding or other interaction of the therapeutic agent and the dressing, inability to use a wide variety of therapeutic agents, and inadequate adhesion of the dressing.

What is needed is a sterile polymer adhesive film that could: eliminate the need for suturing a wound or surgical site, adequately seal a surgical site or wound from the environment to prevent moisture or debris from reaching the site, optionally provide a therapeutic formulation to the site, be biodegradable to eliminate the need to remove the film, and promote directional cellular growth to securely heal the wound.

### BRIEF SUMMARY OF THE INVENTION

The described embodiments relate to a polymer adhesive film having a micro-pattern arranged on a first surface of the polymer adhesive film for application to wounded tissue to promote directional cell growth. The micro-pattern is sized to allow cells of the wounded tissue to grow directionally in one or two directions within the micro-pattern to promote rapid and efficient healing. In various embodiments, the micro-pattern may be formed of micro-tubes, micro-ridges, micro-troughs, or combinations thereof.

The polymer adhesive film may be applied to surgical sites or other wounds to close the wounds and/or cover damaged tissue. The polymer adhesive film may be formulated to adhere to wet tissues such as oral tissues or internal tissues and may be water-proof to prevent water or debris from entering the wound. Furthermore, the polymer adhesive film may be biodegradable to prevent the need to remove the film. The polymer adhesive film may include a therapeutic formulation or pharmaceutical drug to be released over time at the wound or surgical site to promote healing. The polymer adhesive film may be particularly useful for, but is not limited to, closing a surgical site in oral tissue after oral surgical procedures, such as tooth extraction or dental implant insertion.

### BRIEF DESCRIPTION OF THE DRAWINGS

FIG. 1 illustrates a plan view of an embodiment of a polymer adhesive film described herein.

FIG. 2 illustrates a plan view of a second embodiment of a polymer adhesive film described herein.

FIG. 3 illustrates a plan view of a third embodiment of a polymer adhesive film described herein.

FIG. 4 illustrates a perspective view of a portion of the third embodiment of the polymer adhesive film described herein.

FIG. 5 illustrates a cut-away side view of a fourth embodiment of a polymer adhesive film described herein.

FIG. 6 illustrates a cut-away side view of a fifth embodiment of a polymer adhesive film described herein.

FIG. 7 illustrates a cut-away side view of a sixth embodiment of a polymer adhesive film described herein.

FIG. 8 illustrates a cut-away side view of a seventh embodiment of a polymer adhesive film described herein.

FIG. 9 illustrates a cut-away side view of an eighth embodiment of a polymer adhesive film described herein.

FIG. 10 illustrates a cut-away side view of a ninth embodiment of a polymer adhesive film described herein.

FIG. 11 illustrates a cut-away side view of a tenth embodiment of a polymer adhesive film described herein.

FIG. 12 illustrates a cut-away side view of an eleventh embodiment of a polymer adhesive film described herein.

FIG. 13 illustrates a cut-away side view of a twelfth embodiment of a polymer adhesive film described herein.

FIG. 14 illustrates a plan view of a thirteenth embodiment of a polymer adhesive film described herein.

FIG. 15 illustrates a plan view of a fourteenth embodiment of a polymer adhesive film described herein.

FIG. 16 illustrates a perspective view of a portion of a fifteenth embodiment of a polymer adhesive film described herein.

### DETAILED DESCRIPTION OF THE INVENTION

Surgical incisions and other wounds may heal by primary intention or secondary intention. In healing by primary intention, all tissues are brought together and held in place by mechanical means. In contrast, healing by secondary intention occurs when the margins of the wound are not completely approximated (closed), leaving the wound partially open; yet, the wound still heals, albeit through a distinctly different, much slower process (ie. healing from the “bottom up”). Healing by primary intention is preferable to healing by secondary intention because it minimizes the risk of infection, reduces scar tissue formation, minimizes discomfort during healing, and enables faster healing. The polymer adhesive film embodiments described herein may be used to hold together the ends of wounds in various tissues to facilitate healing by primary intention, while the micro-pattern arranged on a first surface of the polymer adhesive film promotes directional cell growth.

Additionally, the polymer adhesive films describe herein are especially advantageous for use in closing surgical sites or wounds in which the edges of the site may not be brought together, for example, in the case of a tooth extraction in which the gap is too large to be completely closed. In this case, the micro-pattern in the polymer adhesive film may promote directional cell growth across the top of the site so that the site behaves as if it were undergoing primary intention, even though all tissues in the site may not be brought together. Thus, the site will heal from the top down and from the bottom up to facilitate faster healing.

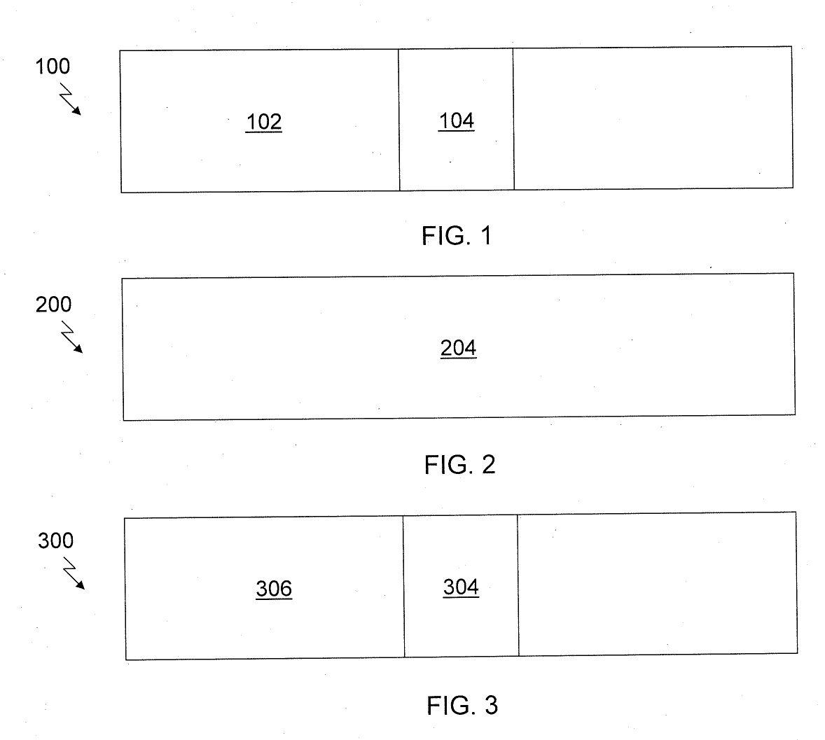

Example embodiments are now described with reference to the accompanying figures wherein like reference numbers are used consistently for like features throughout the drawings. FIG. 1 shows a plan view of an embodiment of a polymer adhesive film 100. The polymer adhesive film 100 includes a micro-patterned portion 104 and non-patterned portions 102 arranged on one side of the polymer adhesive film 100. In use, the micro-patterned portion 104 will be arranged directly on a surgical site or wound and the non-patterned portions 102 will be arranged on either side of the site. As described below in greater detail, the micro-patterns of the micro-patterned portion 104 are arranged to facilitate directional cellular growth along the micro-patterns to heal wounds more quickly.

The polymer adhesive film 100 may be formed of a polymer suitable for use with the specific tissue to which the film 100 is to be applied. For example, the polymer may include various combinations of features such as biocompatibility and biodegradability, mechanical compliance with the specific tissue it is to be used with, strong adhesion under wet or dry conditions as appropriate, elicitation of a minimal inflammatory response, and the ability to deliver therapeutic or pharmaceutical drug formulations. The polymer adhesive film may be formulated from polymers known to adhere to wet tissues, such as oral or internal mucosal tissues, and may be water-proof to prevent water or debris from entering the wound.

In one embodiment, the polymer used to form the polymer adhesive film 100 may include a biodegradable condensation polymer of glycerol and a diacid, such as those described in U.S. Patent Application Publication No. 2003/0118692, the disclosure of which is hereby incorporated by reference in its entirety. For example, the polymer adhesive film 100 may be made up of poly(glycerol sebacate), poly(glycerol sebacate)-acrylate having low acrylation, poly(glycerol sebacate)-acrylate having high acrylation, poly(glycerol sebacate)-acrylate-co-poly(ethylene glycol) networks, poly(glycerol malonate), poly(glycerol succinate), poly(glycerol glutarate), poly(glycerol adipate), poly(glycerol pimelate), poly(glycerol suberate), poly(glycerol azelate), polymers of glycerol and diacids having more than 10, more than 15, more than 20, and more than 25 carbon atoms, polymers of glycerol and non-aliphatic diacids, and mixtures thereof. In various embodiments, amines and aromatic groups, such as terephthalic acid and carboxyphenoxypropane may be incorporated into the carbon chain. The diacids may also include substituents as well, such as amine and hydroxyl, to increase the number of sites available for cross-linking, amino acids and other biomolecules to modify the biological properties of the polymer, and aromatic groups, aliphatic groups, and halogen atoms to modify the inter-chain interactions within the polymer.

The polymer may further include a biomolecule, a hydrophilic group, a hydrophobic group, a non-protein organic group, an acid, a small molecule, a bioactive agent, a controlled-release therapeutic agent or pharmaceutical drug, or a combination thereof. The polymer may be seeded with cells compatible with the tissue that the polymer adhesive film 100 is designed to cover to facilitate rapid healing.

The polymer adhesive film 100 may be coated, for example, by spin coating, with a thin layer of oxidized dextran having aldehyde functionalities (DXTA) to promote covalent cross linking with tissue to which the polymer adhesive film 100 is applied. The terminal aldehyde groups in DXTA react with resident amine groups in proteins forming an imine, while the aldehyde groups of DXTA form a hemiacetal with free hydroxyl groups from a glycerol subunit of the polymer adhesive film 100 surface. The use of DXTA is especially useful to increase the adhesion of the polymer adhesive film 100 to tissue in a wet environment, such as an oral cavity or on internal tissues.

The relative widths of the micro-patterned portion 104 and non-patterned portions 102 may be adjusted to various lengths on of the polymer adhesive film 100 depending on the intended use of the film 100. For example, FIG. 2 shows a plan view of a second embodiment of a polymer adhesive film 200 in which the micro-patterned portion 204 extends over the entire surface of the polymer adhesive film 200. Furthermore, the dimensions of the polymer adhesive films 100, 200 may be modified as needed for a particular application. For example, the overall thickness of the polymer adhesive films of the various embodiments described herein may be adjusted to strike an appropriate balance between the strength and flexibility of the film.

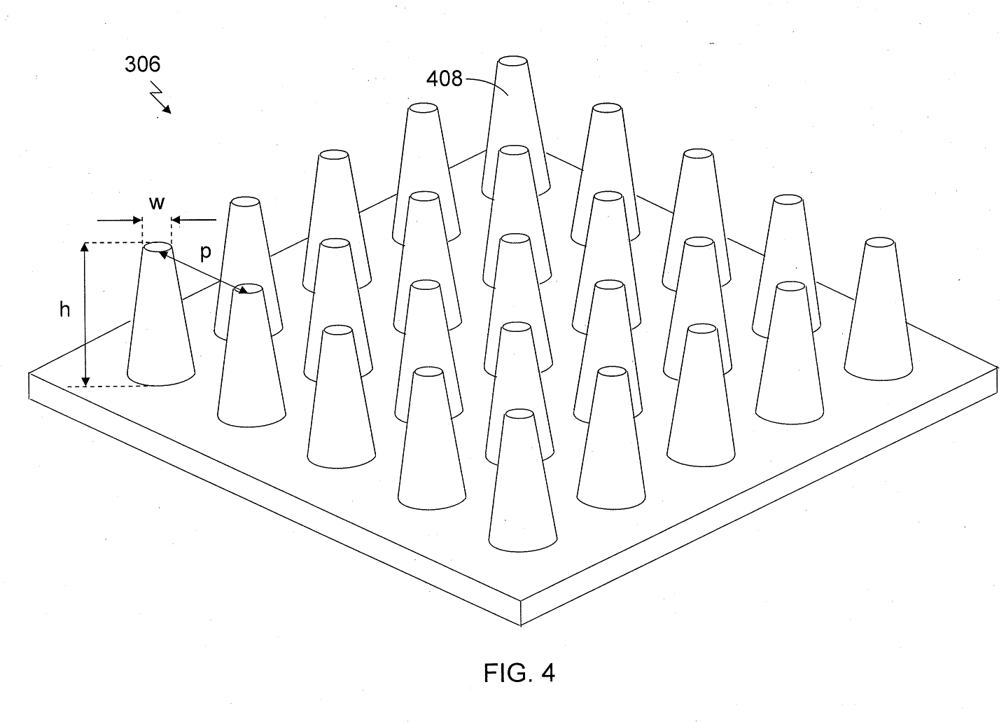

FIG. 3 shows a plan view of a third embodiment of a polymer adhesive film 300 that includes a micro-patterned portion 304 for promoting directional cellular growth and a nano-patterned portion 306 for increasing the adhesion of the polymer adhesive film 300 to the tissue. FIG. 4 shows a perspective view of a portion of the nano-patterned portion 306. As shown in FIG. 4, the nano-patterned portion 306 includes an array of pillars 408 arranged on the surface of the nano-patterned portion 306 of the polymer adhesive film 300. The pillars 408 increase the adhesion of the polymer adhesive film 300 to the tissue by allowing the film 300 to conform and adhere to the uneven surface of the tissue, thus maximizing interfacial contact to enhance adhesion.

A mold used to produce the pillars 408 of the nano-patterned portion 306 may be prepared by patterning a silicon substrate using a combination of photolithography and reactive ion etching to generate the mold. The pillars 408 may then be formed by casting the polymer adhesive film 300 onto the mold and curing the adhesive film 300, for example using ultraviolet light or heat, as appropriate to the particular polymer. The dimensions of the pillars 408, including the tip width w, height h, and pitch p, may vary according to the tissue to which the polymer adhesive film 300 is to be affixed. In one embodiment, the pillars 408 may include tip widths w ranging from about 100 nm to about 1 μm and pillar heights h from about 0.8 μm to about 3 μm. The nano-patterned portion 306 may be coated with a layer of DXTA, as described above, to further improve the adhesion properties of the polymer adhesive film 300.

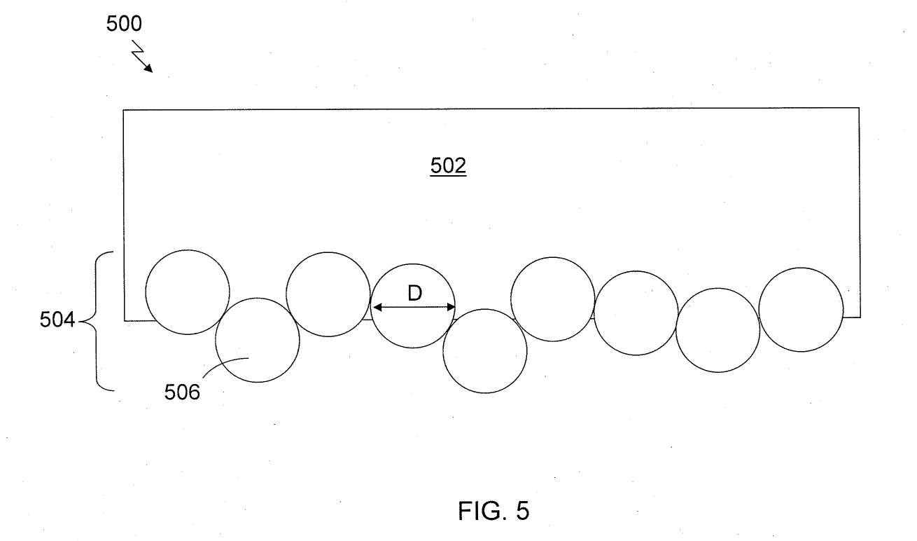

FIG. 5 shows a cut-away side view of a fourth embodiment of a polymer adhesive film 500 made up of a polymer layer 502 and a micro-patterned portion 504 made up of micro-tubes 506 arranged on one side of the polymer layer 502. The micro-patterned portion 504 of the adhesive film 500 may be incorporated as the micro-patterned portion 104, 204, 304 of the polymer adhesive films 100, 200, 300 shown in the embodiments of FIGS. 1-3, respectively. As shown in FIG. 5, the micro-tubes 506 may be closely packed so that the cells of the tissue to be repaired will grow directionally through the micro-tubes 506. When the biodegradable polymer adhesive film 500 disintegrates, the cells will fill the gaps left by the film 500 to complete the healing process.

The micro-tubes 506 may be carbon micro-tubes or any other type of micro-tubes, which are commercially available and preferably purified, for example, single wall micro- or nano-tubes, multi-wall micro- or nano-tubes, bamboo micro- or nano-tubes, and the like. The micro-tubes 506 may be formed of carbon or other materials, which may be biodegradable.

The diameter D of the micro-tubes 506 may be sized to accommodate the type of cells surrounding the wound or site to which the polymer adhesive film 500 will be affixed. The diameter D of the micro-tubes 506 may be as small as the size of at least one biological cell or at least one cell process or may be sized to accommodate the combined size of a group of cells. In various embodiments, the diameter D of the micro-tubes 506 may be between about 0.5 μm to about 100 μm, larger than 100 μm, or between about 10 μm to about 40 μm. The length of the micro-tubes 506 may vary as well, according to the desired application. In various embodiments, the micro-tubes 506 may stretch all the way across a micro-patterned area 104, 204, 304. In other embodiments, the micro-tubes 506 may be shorter than the width of the micro-patterned area 104, 204, 304, and may overlap each other.

In one embodiment, the polymer adhesive film 500 may be formed by forming a polymer layer 502, for example, by casting or extrusion. Next, micro-tubes 506 may be applied to the polymer layer 502 while the polymer layer 502 is in a semi-solid phase, for example, by rolling, spraying, or immersion. The polymer layer 502 may then be rubbed or combed in one direction to align the polymer molecules in the same direction. Physical contact of the polymer molecules with the micro-tubes 506 aligns the micro-tubes 506 in generally the same direction. The polymer layer 502 may then be cured, for example, by ultraviolet light or heating, to lock in the direction of the micro-tubes 506. An additional step of etching back the polymer layer 502 may also be performed to expose larger portions of the micro-tubes 506 so that cells may more easily grow through the tubes.

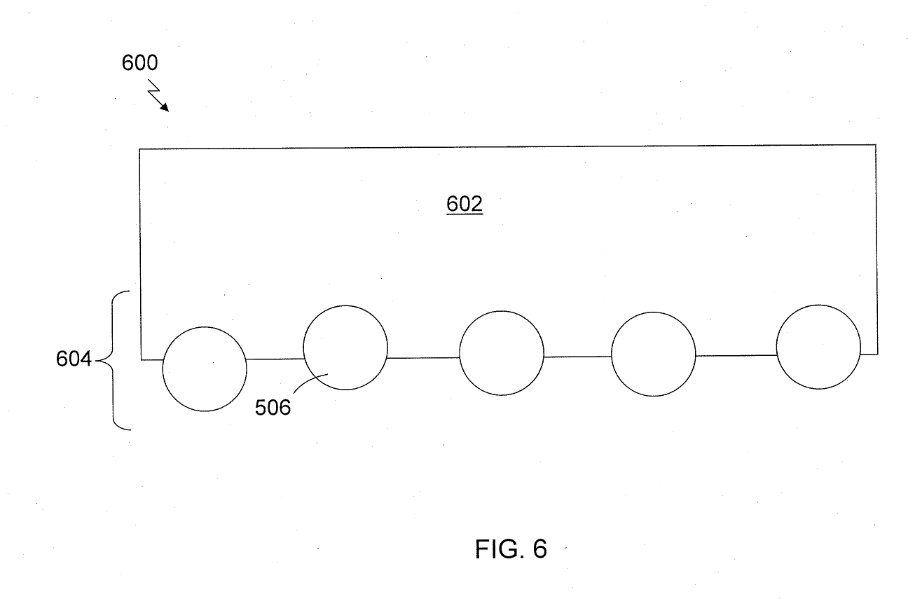

FIG. 6 shows a cut-away side view of a fifth embodiment of a polymer adhesive film 600 made up of a polymer layer 602 and a micro-patterned portion 604 made up of micro-tubes 506 arranged on one side of the polymer layer 602. The polymer adhesive film 600 is similar to the polymer adhesive film 500 of FIG. 5, except that the micro-tubes 506 of polymer adhesive film 600 may be spaced apart so that the cells of the tissue to be repaired will grow directionally both through and between the micro-tubes 506. When the biodegradable polymer adhesive film 600 disintegrates, the cells will fill the gaps left by the film 600 to complete the healing process.

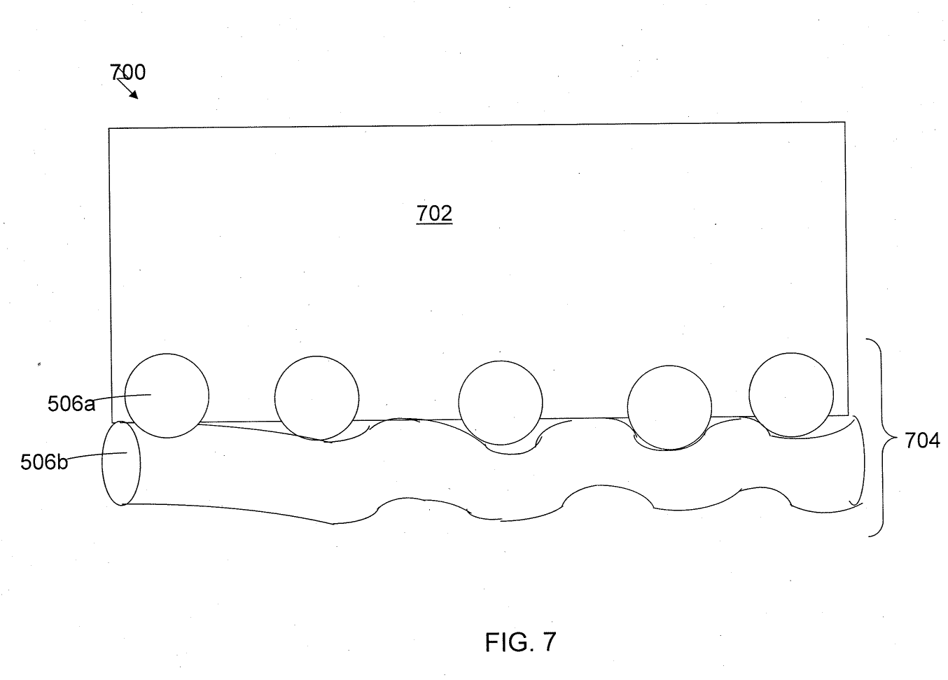

FIG. 7 shows a cut-away side view of a sixth embodiment of a polymer adhesive film 700 made up of a polymer layer 702 and a micro-patterned portion 704 made up of micro-tubes 506a, 506b arranged on one side of the polymer layer 702. The polymer adhesive film 700 is similar to the polymer adhesive film 500 of FIG. 5, except that the micro-tubes 506a, 506b include a first layer of micro-tubes 506a arranged in a first direction, and a second layer of micro-tubes 506b arranged in a second direction perpendicular to the first direction. The perpendicular micro-tubes 506a, 506b will facilitate directional cellular growth in two directions. When the biodegradable polymer adhesive film 700 disintegrates, the cells will fill the gaps left by the film 700 to complete the healing process.

In one embodiment, the polymer adhesive film 700 may be formed by forming a polymer layer 702. Next, micro-tubes 506a may be applied to the polymer layer 702 while the polymer layer 702 is in a semi-solid phase. The polymer layer 702 may then be rubbed or combed in one direction to align the polymer molecules and micro-tubes 506a in the same direction. A second layer of perpendicular directionally oriented polymer and micro-tubes 506b may be overlaid on the first polymer layer 702. The polymer layer 702 may then be cured, and etching back the polymer layer 702 may be performed to expose larger portions of the micro-tubes 506a, 506b.

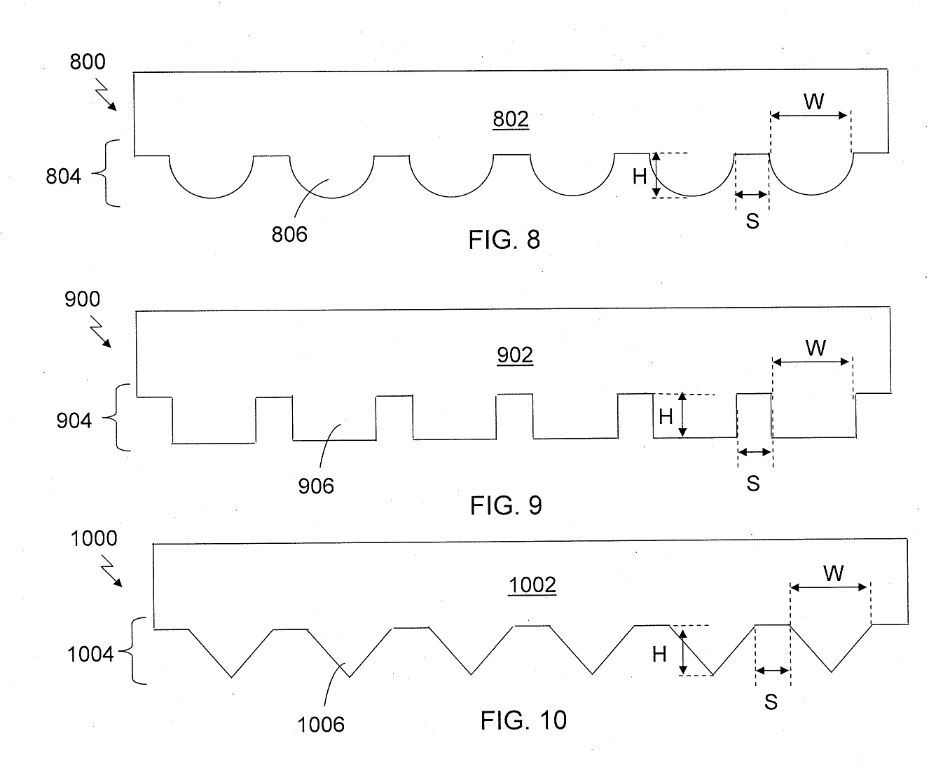

FIG. 8 shows a cut-away side view of a seventh embodiment of a polymer adhesive film 800 made up of a polymer layer 802 and a micro-patterned portion 804 made up of micro-ridges 806 arranged on one side of the polymer layer 802. The micro-patterned portion 804 of the adhesive film 800 may be incorporated as the micro-patterned portion 104, 204, 304 of the polymer adhesive films 100, 200, 300 shown in the embodiments of FIGS. 1-3, respectively. The micro-ridges 806 are arranged parallel to each other and may extend the length of the micro-patterned portion 104, 204, 304. When the polymer adhesive film 800 is applied to a wound or surgery site, the micro-ridges 806 will direct the cell growth between the micro-ridges 806 and across (perpendicular to) the wound or surgical site. When the biodegradable polymer adhesive film 800 disintegrates, the cells will fill the gaps left by the film 800 to complete the healing process.

The micro-ridges 806 may be formed in various geometric or irregular shapes. As shown in FIG. 8, the micro-ridges 806 may have a cross-section shaped as half circles extending from the polymer layer 802. FIG. 9 shows a cut-away side view of an eighth embodiment of a polymer adhesive film 900 made up of a polymer layer 902 and a micro-patterned portion 904 made up of micro-ridges 906 having a cross-sectional shape of a rectangle. FIG. 10 shows a cut-away side view of a ninth embodiment of a polymer adhesive film 1000 made up of a polymer layer 1002 and a micro-patterned portion 1004 made up of micro-ridges 1006 having a cross-sectional shape of a triangle. In various other embodiments, the micro-ridges may have other cross-sectional shapes, such as partial ovals, arcs, trapezoids, squares, irregular polyhedrals, and combinations thereof.

The width of the spacing S between the micro-ridges 806, 906, 1006 may be sized to accommodate the type of cells surrounding the wound or site to which the polymer adhesive film 800, 900, 1000 will be affixed. The spacing S between the micro-ridges 806, 906, 1006 may be as small as the size of at least one biological cell or at least one cell process or may be sized to accommodate the combined size of a group of cells. In various embodiments, the spacing S between the micro-ridges 806, 906, 1006 may be between about 0.5 μm to about 100 μm, larger than 100 μm, or between about 10 μm to about 40 μm. The width W and height H of the micro-ridges 806, 906, 1006 may be varied depending on the application.

In one embodiment, the polymer adhesive films 800, 900, 1000 may be formed by forming a polymer layer 802, 902, 1002, for example, by casting or extrusion. Next, micro-ridges 806, 906, 1006 may be formed on the polymer layer 802, 902, 1002 while the polymer layer 802, 902, 1002 is in a semi-solid phase, for example, by applying a negative micro-mold to the polymer layer 802, 902, 1002. The polymer layer 802, 902, 1002 may then be cured, for example, by ultraviolet light or heating. In various embodiments, the micro-ridges 806, 906, 1006 may be formed by other methods, for example, by a photoresist and etching process.

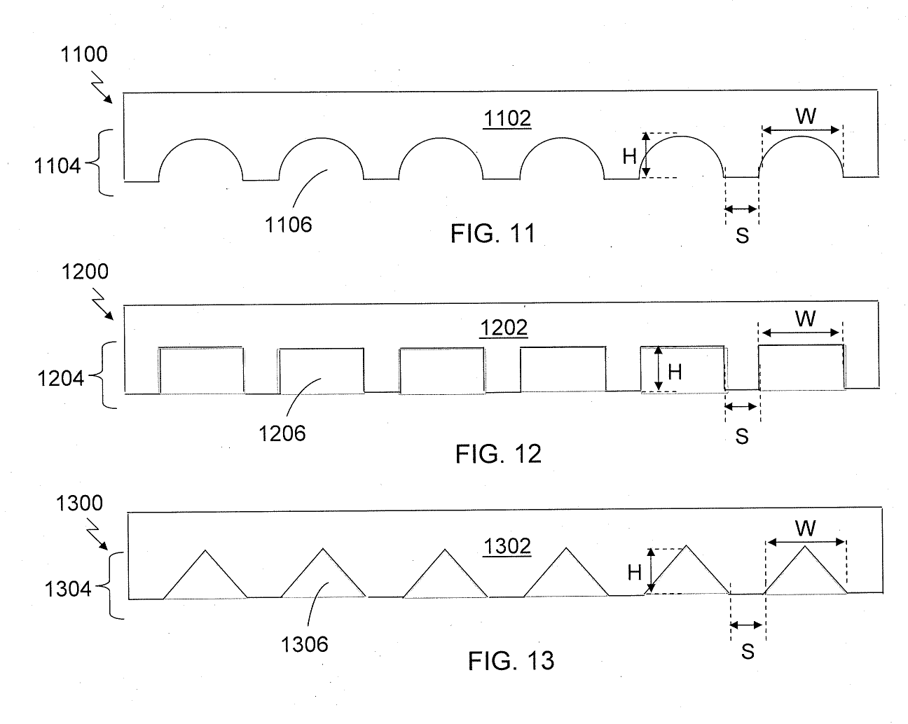

FIG. 11 shows a cut-away side view of a tenth embodiment of a polymer adhesive film 1100 made up of a polymer layer 1102 and a micro-patterned portion 1104 made up of micro-troughs 1106 arranged on one side of the polymer layer 1102. The micro-patterned portion 1104 of the adhesive film 1100 may be incorporated as the micro-patterned portion 104, 204, 304 of the polymer adhesive films 130, 200, 300 shown in the embodiments of FIGS. 1-3, respectively. The micro-troughs 1106 are arranged parallel to each other and may extend the length of the micro-patterned portion 104, 204, 304. When the polymer adhesive film 1100 is applied to a wound or surgery site, the micro-troughs 1106 will direct the cell growth between the micro-troughs 1106 and across (perpendicular to) the wound or surgical site. When the biodegradable polymer adhesive film 1100 disintegrates, the cells will fill the gaps left by the film 1100 to complete the healing process.

The micro-troughs 1106 may be formed in various geometric shapes or irregular shapes. As shown in FIG. 11, the micro-troughs 1106 may have a cross-section shaped as half circles extending into the polymer layer 1102. FIG. 12 shows a cut-away side view of an eleventh embodiment of a polymer adhesive film 1200 made up of a polymer layer 1202 and a micro-patterned portion 1204 made up of micro-troughs 1206 having a cross-sectional shape of a rectangle. FIG. 13 shows a cut-away side view of a twelfth embodiment of a polymer adhesive film 1300 made up of a polymer layer 1302 and a micro-patterned portion 1304 made up of micro-troughs 1306 having a cross-sectional shape of a triangle. In various other embodiments, the micro-troughs may have other cross-sectional shapes, such as partial ovals, arcs, trapezoids, squares, irregular polyhedrals, and combinations thereof.

The width W of the micro-troughs 1106, 1206, 1306 may be sized to accommodate the type of cells surrounding the wound or site to which the polymer adhesive film 1100, 1200, 1300 will be affixed. The width W of the micro-troughs 1106, 1206, 1306 may be as small as the size of at least one biological cell or at least one cell process or may be sized to accommodate the combined size of a group of cells. In various embodiments, the width W between the micro-troughs 1106, 1206, 1306 may be between about 0.5 μm to about 130 μm, larger than 130 μm, or between about 13 μm to about 40 μm. The spacing S between and height H of the micro-troughs 1106, 1206, 1306 may be varied depending on the application.

In one embodiment, the polymer adhesive films 1100, 1200, 1300 may be formed by forming a polymer layer 1102, 1202, 1302, for example, by casting or extrusion. Next, micro-troughs 1106, 1206, 1306 may be formed on the polymer layer 1102, 1202, 1302 while the polymer layer 1102, 1202, 1302 is in a semi-solid phase, for example, by applying a positive micro-mold to the polymer layer 1102, 1202, 1302. The polymer layer 1102, 1202, 1302 may then be cured, for example, by ultraviolet light or heating. In various embodiments, the micro-troughs 1106, 1206, 1306 may be formed by other methods, for example, by a photoresist and etching process.

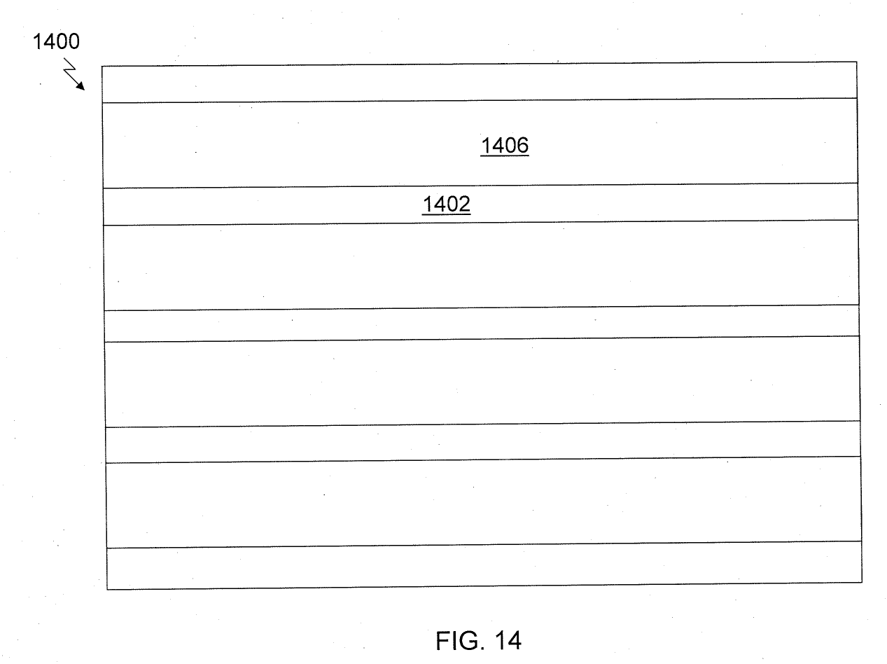

FIG. 14 shows a plan view of a thirteenth embodiment of a polymer adhesive film 1400 including a number of micro-features 1406 arranged parallel to each other on a polymer layer 1402. The micro-features 1406 may be the micro-ridges 806, 906, 1006, or the micro-troughs 1106, 1206, 1306 shown in FIGS. 8-13, respectively. Although the micro-features 1406 of the embodiment of FIG. 14 are shown as having straight sides, in various embodiments, the micro-features could be wavy, jagged, or otherwise shaped.

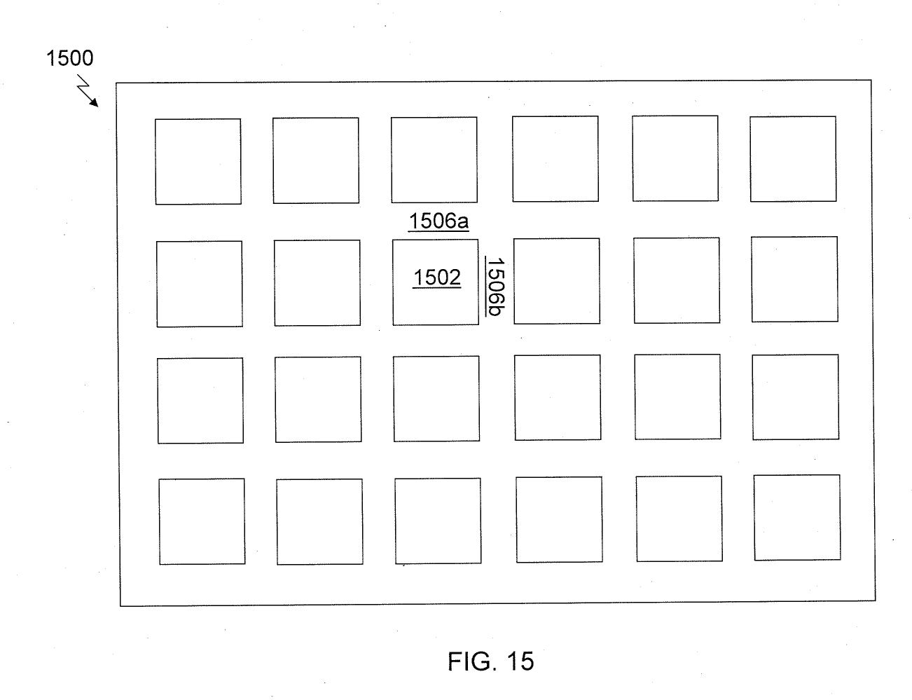

FIG. 15 shows a plan view of a fourteenth embodiment of a polymer adhesive film 1500 including a number of first micro-features 1506 intersecting a number of second micro-features 1506b arranged on a polymer layer 1502. The first micro-features 1506a are arranged parallel to each other and perpendicular to the second micro-features 1506b. The micro-features 1506a, 1506b may be the micro-troughs 1106, 1206, 1306 shown in FIGS. 11-13, respectively. The perpendicular micro-features 1506a, 1506b allow for bi-directional cellular growth both perpendicular and parallel to the wound or surgery site to which the polymer adhesive film 1500 is applied.

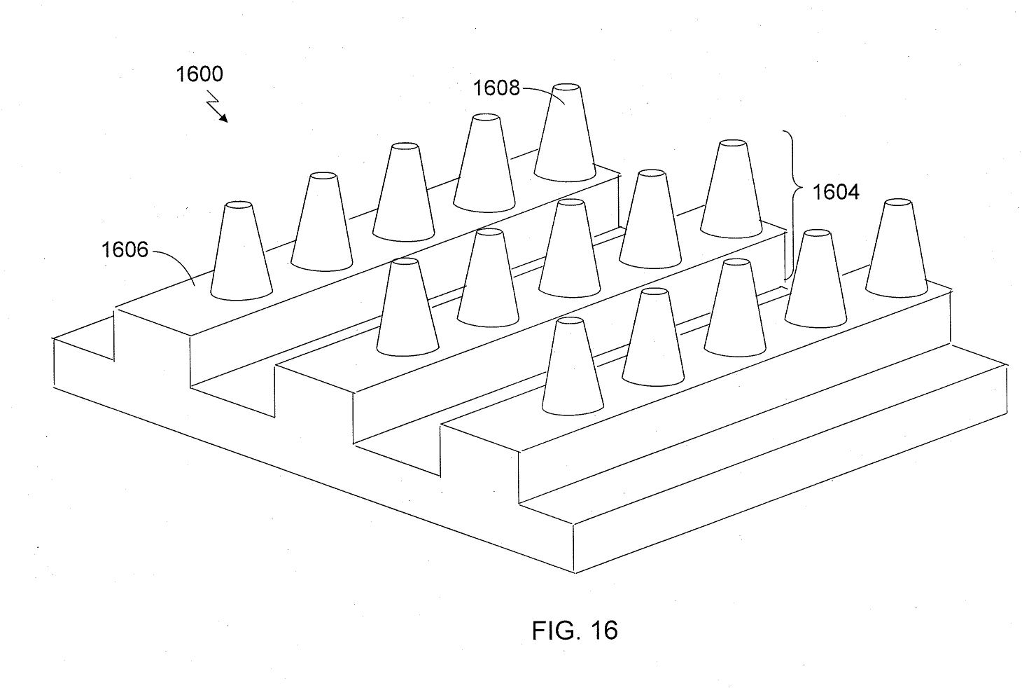

FIG. 16 shows a perspective view of a fifteenth embodiment of a polymer adhesive film 1600 made up of a polymer layer 1602 and a micro-patterned portion 1604 made up of a combination of micro-ridges 1606 and nano-patterned pillars 1608 arranged on one side of the polymer layer 1602. The micro-patterned portion 1604 of the adhesive film 1600 may be incorporated as the micro-patterned portion 104, 204, 304 of the polymer adhesive films 100, 200, 300 shown in the embodiments of FIGS. 1-3, respectively. The micro-ridges 1606 are arranged parallel to each other and may extend the length of the micro-patterned portion 104, 204, 304. The micro-ridges 1606 may be formed in various geometric shapes or irregular shapes, and may be shaped and spaced as the micro-ridges 806, 906, 1006 described in FIGS. 8, 9, and 10, respectively. The pillars 1608 formed as a portion of, or all of, the pillars 408 described in FIG. 4.

When the polymer adhesive film 1600 is applied to a wound or surgery site, the micro-ridges 1606 will direct the cells in directional cellular growth between the micro-ridges 1606 and across, i.e., perpendicular to, the wound or surgery site while the nano-patterned pillars 1608 will increase the adhesion of the polymer adhesive film 1600 to the wound or surgery incision site. When the biodegradable polymer adhesive film 1600 disintegrates, the cells will fill the gaps left by the film 1600 to complete the healing process.

In one embodiment, the polymer adhesive film 1600 may be formed by forming a polymer layer 1602, for example, by casting or extrusion. Next, micro-ridges 1606 and pillars 1608 may be formed on the polymer layer 1602 while the polymer layer 1602 is in a semi-solid phase, for example, by applying a negative micro-mold to the polymer layer 1602. The polymer layer 1602, may then be cured, for example, by ultraviolet light or heating.

Changes and modifications in the specifically described embodiments and methods can be carried out without departing from the scope of the invention which is intended to be limited only by the scope of the appended claims. For example, although the prefixes “micro-” and “nano-” are used in various places throughout the specification and claims, it should be understood that in various embodiments, micro-features could be formed at a nano-scale and vice-versa. Furthermore, it is contemplated that features of the various embodiments could be combined in certain embodiments.

What is claimed as new and desired to be protected by Letters Patent of the United States is:

1. A polymer adhesive film for application to wounded tissue to promote directional cell growth, comprising:
a micro-pattern arranged on a first surface of the polymer adhesive film,
wherein the micro-pattern is sized to allow cells of the wounded tissue to grow directionally within the micro-pattern.

2. The polymer adhesive film of claim 1, wherein the micro-pattern is arranged on only a portion of the first surface of the polymer adhesive film.

3. The polymer adhesive film of claim 1, wherein the micro-pattern comprises a plurality of micro-tubes.

4. The polymer adhesive film of claim 3, wherein the micro-pattern comprises a first plurality of micro-tubes arranged generally in a first direction.

5. The polymer adhesive film of claim 4, wherein the micro-pattern further comprises a second plurality of micro-tubes arranged generally in a second direction, wherein the second direction is generally perpendicular to the first direction.

6. The polymer adhesive film of claim 3, wherein the micro-tubes have a diameter larger than the size of a single cell of the wounded tissue.

7. The polymer adhesive film of claim 3, wherein the micro-tubes have a diameter of between about 0.5 μm to about 100 μm.

8. The polymer adhesive film of claim 1, wherein the micro-pattern comprises a plurality of micro-ridges.

9. The polymer adhesive film of claim 8, wherein the micro-ridges have a cross-sectional shape of a rectangle, a square, a portion of a circle, a portion of an oval, or a triangle.

10. The polymer adhesive film of claim 8, wherein the micro-ridges are spaced apart from each other at a distance that is larger than the size of a single cell of the wounded tissue.

11. The polymer adhesive film of claim 8, wherein the micro-ridges are spaced apart from each other at a distance of between about 0.5 μm to about 100 μm.

12. The polymer adhesive film of claim 8, wherein the micro-ridges further comprise a plurality of pillars arranged on each of the micro-ridges.

13. The polymer adhesive film of claim 1, wherein the micro-pattern comprises a plurality of micro-troughs.

14. The polymer adhesive film of claim 13, wherein the plurality of micro-troughs comprises a first plurality of micro-troughs arranged perpendicularly to a second plurality of micro-troughs.

15. The polymer adhesive film of claim 13, wherein the micro-troughs have a cross-sectional shape of a rectangle, a square, a portion of a circle, a portion of an oval, or a triangle.

16. The polymer adhesive film of claim 13, wherein the micro-troughs have a width that is larger than the size of a single cell of the wounded tissue.

17. The polymer adhesive film of claim 13, wherein the micro-troughs have a width that is between about 0.5 μm to about 100 μm.

18. The polymer adhesive film of claim 1, wherein the polymer adhesive film comprises a biodegradable condensation polymer of glycerol and a diacid.

19. The polymer adhesive film of claim 18, wherein the polymer adhesive film comprises a polymer selected from the group consisting of poly(glycerol sebacate), poly(glycerol sebacate)-acrylate having low acrylation, poly(glycerol sebacate)-acrylate having high acrylation, poly(glycerol sebacate)-acrylate-co-poly(ethylene glycol) networks, poly(glycerol malonate), poly(glycerol succinate), poly(glycerol glutarate), poly(glycerol adipate), poly(glycerol pimelate), poly(glycerol suberate), poly(glycerol azelate), a polymer of glycerol and a diacid having more than 10 carbon atoms, a polymer of glycerol and a diacid having more than 15 carbon atoms, a polymer of glycerol and a diacid having more than 20 carbon atoms, a polymer of glycerol and a diacid having more than 25 carbon atoms, and a polymer of glycerol and a non-aliphatic diacid.

20. The polymer adhesive film of claim 1, wherein the tissue is gingival tissue.

21. A method of forming a polymer adhesive film for application to wounded tissue to promote directional cell growth, the method comprising:
forming a micro-pattern arranged on a first surface of the polymer adhesive film,
wherein the micro-pattern is sized to allow cells of the wounded tissue to grow directionally within the micro-pattern.

22. The method of claim 21, wherein the micro-pattern comprises a plurality of micro-tubes.

23. The method of claim 21, wherein forming the micro-pattern comprises applying a first plurality of micro-tubes to a first polymer adhesive film and curing the first polymer adhesive film.

24. The method of claim 23, further comprising etching the first polymer adhesive film to expose the first plurality of micro-tubes.

25. The method of claim 23, further comprising generally aligning the first plurality of micro-tubes in a first direction.

26. The method of claim 25, further comprising rubbing the first polymer adhesive film to generally align the first plurality of micro-tubes in the first direction.

27. The method of claim 26, further comprising applying a second plurality of micro-tubes to a second polymer adhesive film, rubbing the second polymer adhesive film to generally align the second plurality of micro-tubes in a second direction, and applying the second polymer adhesive film to the first polymer adhesive film, wherein the first direction is generally perpendicular to the second direction.

28. The method of claim 21, wherein the micro-pattern comprises a plurality of micro-ridges.

29. The method of claim 28, wherein the micro-ridges further comprise a plurality of pillars arranged on each of the micro-ridges.

30. The method of claim 28, wherein the micro-troughs are formed using a mold.

31. The method of claim 28, wherein the micro-pattern comprises a plurality of micro-troughs.

32. The method of claim 31, wherein the micro-troughs are formed using a mold.
